(19) United States
(12) Patent Application Publication
(10) Pub. No.: US 2005/0118718 A1
(43) Pub. Date: Jun. 2, 2005

(54) Stabilization and controlled delivery of ionic biopharmaceuticals

(75) Inventors: You Han Bae, Salt Lake City, UT (US); Jong Ho Kim, Salt Lake City, UT (US); Ajay Taluja, Salt Lake City, UT (US)

Correspondence Address: ALAN J. HOWARTH, P.O. BOX 1909, SANDY, UT, 84091-1909, US

(73) Assignee: University of Utah Research Foundation

(21) Appl. No.: 10/948,077

(22) Filed: Sep. 22, 2004

Related U.S. Application Data
(60) Provisional application No. 60/505,055, filed on Sep. 22, 2003.

(51) Int. Cl. C12N 15/88; C07H 21/04; C07K 14/47

(52) U.S. Cl. 435/458; 530/350; 536/23.2; 525/54.1; 525/54.2

(57) ABSTRACT

Compositions and methods for stabilization and controlled release of ionic biopharmaceuticals are disclosed. Illustrative compositions according to the present invention include polyelectrolyte-PEG diblock copolymers complexed by ionic bonds with the ionic biopharmaceuticals. Additional illustrative compositions include microspheres encapsulating polyelectrolyte-PEG diblock copolymers complexed by ionic bonds with the ionic biopharmaceuticals. A method of delivering a biopharmaceutical to a person in need thereof involves administering microspheres that encapsulate biopharmaceutical/polyelectrolyte-PEG diblock copolymer complexes.

### CROSS-REFERENCE TO RELATED APPLICATIONS

This application claims the benefit of U.S. Provisional Application No. 60/505,055, filed Sep. 22, 2003, which is hereby incorporated by reference.

### STATEMENT REGARDING FEDERALLY SPONSORED RESEARCH OR DEVELOPMENT

Not Applicable.

### BACKGROUND OF THE INVENTION

This invention relates to drug delivery. More particularly, this invention relates to stabilization and delivery of ionic macromolecular drugs.

The revolutionary potential of products derived from molecular biology for health care has opened new frontiers in pharmaceutical applications. The exponential growth of biotechnology-derived drug products and potential candidates in the health care sector has been driven by mind-boggling research in recombinant DNA technology. M. M. Struck, Biopharmaceutical R&D success rates and development times—A new analysis provides benchmarks for the future, 12 Biotechnology 674-677 (1994). As the skills of molecular biologists expand to produce more recombinant protein drugs and those of biochemists increase to produce purer products, pharmaceutical scientists are faced with greater and more complex formulation challenges. J. L. Cleland & R. Langer, Formulation and Delivery of Proteins and Peptides (American Chemical Society, Washington D.C. 1994). Despite recent progress in biotechnology, two problems continue to hinder the use of biological macromolecules in medicine and industry: (1) molecular stability and sensitivity of higher order tertiary structures to chemical and physical stresses during manufacture, storage, and drug delivery, and (2) delivery of therapeutic macromolecules requires vehicles and carriers that release native proteins, enzymes, antibodies, hormones, nucleic acids, and peptides at a rate that is consistent with the needs of particular patients or treatment of the disease process.

Concerning the first of these problems, macromolecular stability, numerous factors differentiate biological macromolecules from conventional chemical entities, for example, their size, conformation, and amphiphilic nature. S. S. Davis & L. Illum, Drug delivery for challenging molecules-Commentary, 176 International Journal of Pharmaceutics 1-8 (1998). Macromolecules are not only susceptible to chemical, but also physical degradation. They are sensitive to a variety of environmental factors such as temperature, oxidizing agents, pH, freezing, shaking, and shear stress. M. C. Manning et al., Stability of Protein Pharmaceuticals-Review, 6 Pharmaceutical Research 903-918 (1989). In considering a macromolecule for drug development, stability factors must be actively considered when choosing a production process. Maintenance of biological activity during the development and manufacture of pharmaceutical products depends on the inherent stability, as well as the stabilization techniques used.

Destabilization of protein and peptides molecules is of two types: chemical, which involves modifications in covalent bonds, and physical, which involves changes in spatial, three-dimensional structure (i.e., denaturation). The chemical degradation pathways include hydrolysis, oxidation, deamidation, disulfide exchange, and racemization. T. H. Nguyen et al., The kinetics of relaxin oxidation by hydrogen peroxide, 10 Pharmaceutical Research 1563-1571 (1993); S. Li et al., Aggregation and precipitation of human relaxin induced by metal-catalyzed oxidation, 34 Biochemistry 5762-5772 (1995); S. Li et al., Chemical Pathways of peptide degradation. V. Ascorbic acid promotes rather than inhibits the oxidation of methionine to methionine sulfoxide in small model peptides, 10 Pharmaceutical Research 1572-1579 (1993); K. Patel & R. T. Borchardt, Chemical Pathways of peptide degradation. II. Kinetics of deamidation of an aspararaginyl residue in a model hexapeptide, 7 Pharmaceutical Research 703-711 (1990); K. Mehrnaz & R. T. Borchardt, Chemical Pathways of peptide degradation. IX. Metal catalyzed oxidation of histidine in model peptides, 15 Pharmaceutical Research 1096-1101 (1998); T. Geiger & S. Clarke, Deamidation, isomerization and racemization at asparaginyl and aspartyl residues in peptides, 262 J. Biol. Chem. 785-794 (1987). The physical or denaturation process is unfolding of the molecule, resulting in problems of aggregation, adsorption, and loss of activity, and greatly concerns formulation scientists. M. C. Manning et al., supra. Tertiary structure of the protein that must be stabilized against the various disruptive forces that occur during processing and handling. Potential denaturing forces include chemical stress from factors used in purification, such as pH, ionic strength, or detergents, or physical stress during manufacture processes, where surface adsorption and shear contribute to unwinding of the tertiary structure into a random coil.

Concerning the second of these problems, delivery, the therapeutic and commercial success of protein or peptide drugs developed from biotechnology research depends in part on the ability to formulate and deliver these drugs. J. L. Cleland & R. Langer, supra. While the technology exists for the discovery and development of such drugs, several challenges need to be met with regard to their delivery in convenient, controlled release, and targeted formulations. The most convenient route for the systemic delivery of pharmaceuticals is oral. However, attempts to deliver large molecular weight proteins and peptides orally have not been widely successful. Bioavailability via this route is poor for molecules of molecular mass greater than several hundred daltons. In addition, proteins are susceptible to hydrolysis and modification at gastric pH and can be degraded by proteolytic enzymes in the small intestine. J. P. Bai & L. L. Chang, Comparison of site-dependent degradation of peptide drugs within the gut of rats and rabbits, 45 J. Pharm. Pharmacol. 1085-2087 (1993). Recently, there has been interest in the use of biodegradable polymer systems for controlled release of proteins. D. L. Wise et al., Opportunities and challenges in the design of implantable biodegradable polymeric systems for the delivery of antimicrobial agents and vaccines, 1 Adv. Drug Delivery Rev. 19-39 (1987); S. Cohen et al., Novel approaches to controlled-release antigen delivery, 10 Int. J. Tech. Assoc. Health Care 121-130 (1994). In these applications, protein drugs are embedded in polymer matrices that undergo hydrolysis or enzymatic digestion, resulting in controlled release of the protein. Polymers have also been widely investigated for use in protein-polymer conjugates. These systems have generally been utilized for prolonging the circulation half-lives of proteins or for delivering targeted payloads of protein pharmaceuticals to specific tissues. A major obstacle to development of these polymers is the need to retain the structure and biological activity of encapsulated proteins during period of incubation under physiological conditions.

PEGylation is the covalent attachment of polyethylene glycol (PEG) for modifying biological macromolecules, peptides, and proteins. J. M. Harris, Polyethylene Glycol Chemistry, Biotechnical and Biomedical Applications (Plenum, New York 1992). PEGylation raises the molecular weight of proteins and shields antigenic and immunogenic epitopes, shields receptor-mediated uptake by the reticuloendothelial system (RES), and prevents recognition and degradation by proteolytic enzymes. PEGylation reduces renal filtration and alters biodistribution, since it increases the apparent size of the proteins. M. J. Roberts et al., Chemistry for peptide and protein PEGylation, 54 Adv. Drug Delivery Rev. 459-476 (2002). However, the bioactivity of a PEGylated protein is influenced by the location of the PEG sites on the protein and the number and length of PEG chains attached to the protein. In the case of insulin, mPEG-PheB1-insulin conjugates were substantially more stable than native insulin, but mPEG-LysB29-insulin conjugates were only slightly more stable than native insulin. F. Liu et al., Glucose-induced release of glycosylpoly(ethylene glycol) insulin bound to a soluble conjugate of concanavalin A, 8 Bioconjugate Chem. 664-672 (1997). It is very difficult to attach PEG at a targeted site on a protein such that biological activity of the protein is not altered. In addition, this approach requires complicated procedures to separate selected PEGylated proteins from unselected PEGylated proteins. D. H. Na et al., Identification of the modifying sites of mono-PEGylated salmon calcitonins by capillary electrophoresis and MALDI-TOF mass spectrometry, 754 J. Chromatogr. B Biomed. Sci. Appl. 259-263 (2001).

One method available to extend the delivery of proteins that are difficult to deliver or inconvenient to repetitively administer is encapsulating the protein in microspheres comprising a biodegradable polymer. S. Cohen et al., Controlled delivery systems for proteins based on poly(lactic/glycolic acid) microspheres, 8 Pharmaceutical Research 713-720 (1991). Microspheres comprising a drug dispersed or encapsulated in a polymer have been developed for use in medicine. Among the different encapsulation techniques, the multiple emulsion method is generally considered as one of the most convenient ways to encapsulate water soluble proteins. Y. Ogawa et al., In vivo release profiles of leuprolide acetate from microcapsules prepared with polylactic acids of copoly(lactic/glycolic) acids and in vivo degradation of these polymers, 36 Chemical & Pharmaceutical Bulletin 2576-2581 (1988); H. Okada et al., Pharmacokinetics of once-a-month injectable microspheres of leuprolide acetate, 8 Pharmaceutical Research 787-791 (1991). Microspheres comprising poly(caprolactone) (PCL), poly(lactide) (PLA), poly(glycolide) (PGA), and their copolymers, have been studied extensively as protein delivery systems. Y. Y. Yang et al., Effect of preparation temperature on the characteristics and release profiles of PLGA microspheres containing protein fabricated by double-emulsion solvent extraction/evaporation method, 69 J. Control. Release 81-96 (2000); J. Pean et al., Why does PEG 400 co-encapsulation improve NGF stability and release from PLGA biodegradable microspheres, 16 Pharmaceutical Research 1294-1299 (1999). The release profile of protein from these microspheres has a tri-phasic pattern caused by low diffusivity of the protein and a slow erosion rate of the polymers. To increase the diffusivity of the protein and degradation of the polymers, a hydrophilic material such as poly(ethylene glycol) (PEG) was introduced into microsphere formulations. J. M. Bezemer et al., Control of protein delivery from amphiphilic poly(ether ester) multiblock copolymers by varying their water content using emulsification techniques, 66 J. Control. Release 307-320 (2000); J. M. Bezemer et al., Microspheres for protein delivery prepared from amphiphilic multiblock copolymers 2. Modulation of release rate, 67 J. Control. Release 249-260 (2000); X. Li et al., Influence of process parameters on the protein stability encapsulated in poly-CL-lactide-poly(ethylene glycol)microspheres, 68 J. Control. Release 41-52 (2000). Recently, poly(lactide-co-glycolide) (PLGA) has been widely used in microspheres for protein delivery because of its biocompatibility. However, PLGA microspheres induce acidic microenvironmental conditions, which easily denature proteins by formation of insoluble aggregates. Recently, research has been carried out concerning the stabilization of proteins against acidic microenvironments. J. Wang et al., Characterization of the initial burst release of a model peptide from poly(D,L-lactide-co-glycolide) microspheres, 82 J. Control. Release 289-307 (2002). To neutralize the acidic microenvironment, magnesium hydroxide (Mg(OH)₂), a poorly soluble base, was added to the PLGA microspheres system. This resulted in an improvement in the release and stability of encapsulated proteins. G. Zhu et al., Stabilization of proteins encapsulated in injectable poly(lactide-co-glycolide), 18 Nature Biotechnology 52-57 (2000).

At the most basic level, gene therapy can be described as the intracellular delivery of genetic material (nucleic acid) to generate a therapeutic effect by correcting an existing abnormality or providing cells with a new function. Gene therapy was originally conceived as a specific gene replacement therapy for correcting heritable defects by delivering functionally active therapeutic genes into targeted cells. A purpose of gene delivery is to deliver plasmid DNA into cells to elicit therapeutic effects, such as induction, enhancement, or blocking of protein expression.

Perhaps one of the greatest problems associated with currently devised gene therapies, whether ex vivo or in vivo, is the inability to transfer DNA efficiently into a targeted cell population and to achieve high level expression of the gene product in vivo. T. Friedmann, Overcoming the Obstacles to Gene Therapy, 276 Scientific American 96-101 (1997). Among the physiological phenomena that inhibit administration of a nucleic acid to an animal tissue are inability to direct the nucleic acid to cells of the selected tissue, inability of the nucleic acid to cross membranes of cells of the selected tissue, nucleolytic digestion of the nucleic acid prior to its delivery to cells of the selected tissue, nucleolytic digestion of the nucleic acid within cells of the selected tissue prior to transfer of the nucleic acid to a location within the cells at which the nucleic acid may exert its intended effect, clearance of the nucleic acid from the animal's system before the nucleic acid has been delivered to a sufficient fraction of cells of the selected tissue, and inability to achieve an adequate dosage of the nucleic acid at the selected tissue.

Viral vectors are regarded as the most efficient system for delivery of nucleic acid in gene therapy, and recombinant, replication-defective viral vectors have been used to transduce (via infection) cells both ex vivo and in vivo. S. L. Brody & R. G. Crystal, R. G, Adenovirus-mediated in vivo gene transfer, 716 Annals N.Y. Acad. Sci. 90-102 (1994); F. L. Cosset & S. J. Russel, Targeting retrovirus entry, 3 Gene Therapy 946-956 (1996); J. Y. Dong et al., Systematic Analysis of Repeated Gene Delivery into Animal Lungs with a Recombinant Adenovirus Vector, 7 Hum. Gene Ther. 319-331 (1996); S. Toggas et al., Central nervous system damage produced by expression of the HIV-1 coat protein gp120 in transgenic mice, 367 Nature 188-193 (1994). Such vectors have included retroviral, adenoviral and adeno-associated, and herpes viral vectors. While highly efficient at gene transfer, the major disadvantages associated with the use of viral vectors include the inability of many viral vectors to infect non-dividing cells; problems associated with insertional mutagenesis; inflammatory reactions to the virus and potential helper virus production; antibody responses to the viral coats; and the potential for production and transmission of harmful virus to other human patients. The efficiency of gene transfer into cells directly influences the resultant gene expression levels.

Due to these disadvantages, improved methods of gene delivery are needed. Such methods should be flexible enough for use with virtually any gene of interest and should permit the introduction of genetic material into a variety of cells and tissues. Non-viral methods represent only a fraction of the methods used in the gene delivery field, but they are catching up with methods involving viral vectors. The use of nonviral vectors is an attractive in vivo gene delivery strategy that is simpler than viral systems and lacks some of their inherent risks. M. Wolfert, et al., Characterization of Vectors for Gene Therapy Formed by Self-Assembly of DNA with Synthetic Block Co-Polymers, 7 Human Gene Therapy 2123-2133 (1996); B. Abdallah et al., Non-viral gene transfer: applications in developmental biology and gene therapy-Review, 85 Biol Cell 1-7 (1995); F. Liu & L. Huang, Development of non-viral vectors for systemic gene delivery, 78 Journal of Controlled Release 259-266 (2002); J. T. Godbey & A. G. Mikos, Recent progress in gene delivery using non-viral transfer complexes, 72 Journal of Controlled Release 115-125 (2001). Liposomes and receptor-mediated polycation systems are promising carriers for delivery and expression of plasmid DNA encoding genes into the target cells.

Most agents suitable for use as condensing agents for polyanionic bioactive agents are polycations. C. W. Pouton & L. W. Seymour, Key issues in non-viral gene delivery, 46 Advanced Drug Delivery Reviews 187-203 (2001); M. E. Davis, Non-viral gene delivery systems, 13 Current Opinion in Biotechnology 128-131 (2002); D. Ferber, Gene Therapy: Safe & Virus-Free “New vectors aim to mimic viral vectors pros without their dangerous cons”—News focus, 294 Science 1638-1642 (2001). The term “polycations” generally refers to molecules with more than one positive charge that are able to condense polyanionic bioactive agents such as DNA.

Polylysine and other polypeptides are polycationic polypeptides that have been used as condensing agents for delivery of macromolecules. Several amino acids are known to be positively charged at physiological pH. V. S. Trubetskoy et al., Use of N-terminal modified poly(L-lysine)-antibody conjugate as a carrier for targeted gene delivery in mouse lung endothelial cells, 3 Bioconjug Chem. 323-327 (1992); M. Hashida et al., Targeted delivery of plasmid DNA complexed with galactosylated poly(L-lysine), 53 J. Controlled Release 301-310 (1998); J. M. Benns et al., pH-Sensitive Cationic Polymer Gene Delivery Vehicle: N-Ac-poly(L-histidine)-graft-poly(L-lysine) Comb Shaped Polymer, 11 Bioconjug. Chem. 637-645 (2000); P. Midoux & M. Monsigny, Efficient gene transfer by histidylated polylysine/pDNA complexes, 10 Bioconjug. Chem. 406-411 (1999). Among the naturally occurring, genetically encoded amino acids, lysine, arginine, and histidine are positively charged. Other, naturally occurring non-genetically-encoded amino acids and synthetic amino acids may also be positively charged, as may be other naturally occurring, genetically encoded amino acids under certain conditions. These amino acids can be polymerized into chains, resulting in polycationic polypeptides, which are excellent condensing agents (indeed, polylysine is one specific member of the family of polycationic polypeptides). They may be either homopolymers, such as polylysine, polyarginine, polyornithine, or polyhistidine, or heteropolymers, such as myelin basic protein. One illustrative family of condensing agents is the polylysines. Polylysines comprise chains of varying lengths of positively charged lysine residues. These lysine residues can be either in the D or L configuration, or a mixture of the two enantiomers; poly-L-lysine is illustrative.

The unique chemical properties of polyethylenimine (PEI) underscore its potential as a vector for gene delivery. M. A. Zanta et al., In vitro gene delivery to hepatocytes with galactosylated polyethylenimine, 8 Bioconjug. Chem. 839-844 (1997); M. Ogris et al., PEGylated DNA/transferrin-PEI complexes: reduced interaction with blood components, extended circulation in blood and potential for systemic gene delivery, 6 Gene Ther. 595-605 (1999); W. T. Godbey et al., Poly(ethylenimine)-mediated transfection: A new paradigm for gene delivery, 51 J. Biomed. Mater. Res. 321-328 (2000); O. Boussif et al., A versatile vector for gene and oligonucleotide transfer into cells in culture and in vivo—polyethylenimine, 92 Proc. Nat'l Acad. Sci. USA 7297-7301 (1995). For example, PEI has a very high cationic charge density, making it useful for binding anionic DNA within the physiological pH range and forcing the DNA to form condensates small enough to be effectively endocytosed, which is the primary mode of entry of PEI/DNA complexes into cells via the endosomal compartment, from which PEI/DNA complexes travel to the nucleus. Another property of PEI that makes it suitable as a DNA vector is its structure, in which every third atom of it backbone is a protonatable amino nitrogen, which allows the polymer to function as an effective buffering system for the sudden decrease in pH from the extracellular environment to the endosomal/lysosomal compartment. This feature is important for the protection of genetic material as it travels to the nucleus.

Cationic lipids are able to interact spontaneously with negatively charged DNA to form clusters of aggregated vesicles along the nucleic acid. K. Crook et al., Plasmid DNA-molecules complexed with cationic liposomes are protected from degradation by nucleases and shearing by aerosolisation, 3 Gene Therapy 834-839 (1996); P. L. Felgner, Improved cationic lipid formulations for in vivo gene therapy, 2 Gene Therapy 573 (1995); D. M. Geddes, Liposome mediated gene therapy for cystic fibrosis, 2 Gene Therapy 586 (1995); F. Liu et al., Factors controlling the efficiency of cationic lipid-mediated transfection in vivo via intravenous administration, 4 Gene Therapy 517-523 (1997); D. L. Reimer et al., Formation of novel hydrophobic complexes between cationic lipids and plasmid DNA, 34 Biochemistry 12877-12883 (1995). At a critical liposome density the DNA is condensed and becomes encapsulated within a lipid bilayer, although there is also some evidence that cationic liposomes do not actually encapsulate the DNA, but instead bind along the surface of the DNA, maintaining its original size and shape. Cationic liposomes are also able to interact with negatively charged cell membranes more readily than classical liposomes. Fusion between cationic vesicles and cell surfaces might result in delivery of the DNA directly across the plasma membrane. This process bypasses the endosomal-lysosomal route, which leads to degradation of anionic liposome formulations. Cationic liposomes can be formed from a variety of cationic lipids, and they usually incorporate a neutral lipid such as dioleoylphosphatidyl-ethanolamine (DOPE) into the formulation to facilitate membrane fusion. A variety of cationic lipids have been developed to interact with DNA, but perhaps the best known are N-1(-(2,3-dioleoyloxy)propyl)-N,N,N-trimethylammoniumethyl sulphate (DOTAP) and N-(1-(2,3-dioleoyloxy)propyl)-N,N,N-trimethylammonium chloride (DOTMA). These are commercially available lipids that are sold as in vitro transfecting agents, with the latter sold as Lipofectin™.

Thus, while prior art delivery systems are known and are generally suitable for their limited purposes, they possess certain inherent deficiencies that detract from their overall utility for stabilization and delivery of macromolecules.

In view of the foregoing, it will be appreciated that providing compositions and methods for stabilization and controlled delivery of ionic macromolecules would be a significant advancement in the art.

### BRIEF SUMMARY OF THE INVENTION

An illustrative advantage of the present invention comprises providing compositions and methods for the stabilization and storage of biologically active macromolecules, such as proteins and peptides, nucleic acids, and antisense oligonucleotides (ODNs).

Another advantage is providing for controlled release of biologically active macromolecules, such as proteins and peptides, nucleic acids, and antisense oligonucleotides (ODNs), by encapsulation in suitable formulations and compositions.

These and other advantages are addressed by providing a composition comprising an ionic complex of (a) a biological macromolecule having a charge, and (b) a polyelectrolyte-poly(ethylene glycol) diblock copolymer having an opposite charge. The polyelectrolyte-poly(ethylene glycol) diblock copolymer comprises either a polycation-poly(ethylene glycol)diblock copolymer or a polyanion-poly(ethylene glycol)diblock copolymer. An illustrative polyelectrolyte-poly(ethylene glycol)diblock copolymer comprises a poly(L-histidine)-poly(ethylene glycol)diblock copolymer.

Another illustrative embodiment of the invention comprises a composition comprising a microsphere encapsulating an ionic complex of (a) a biological macromolecule having a charge, and (b) a polyelectrolyte-poly(ethylene glycol)diblock copolymer having an opposite charge. The microsphere comprises a biocompatible polymer, such as poly(lactide-co-glycolide) (PLGA).

Still another illustrative embodiment of the invention comprises a method of stabilizing an ionic macromolecule against inactivation comprising:
(a) determining the charge of the ionic macromolecule; and
(b) complexing the ionic macromolecule with a polyelectrolyte-poly(ethylene glycol)diblock copolymer of opposite charge.

Yet another illustrative embodiment of the invention comprises a method of delivering an ionic macromolecule to an individual in need thereof comprising administering to the individual a pharmaceutically effective amount of a microsphere encapsulating an ionic complex of the ionic macromolecule and a polyelectrolyte-poly(ethylene glycol)diblock copolymer.

Another illustrative embodiment of the invention comprises a method of increasing transfection efficiency of DNA into mammalian cells, the method comprising:
(a) forming an ionic complex comprising the DNA and a cationic polyelectrolyte-poly(ethylene glycol)diblock copolymer; and
(b) incubating the ionic complex with the mammalian cells in the presence of a transfection enhancer such that the ionic complex, and hence the DNA, enters the mammalian cells. An illustrative cationic polyelectrolyte-poly(ethylene glycol)diblock copolymer according to the present invention comprises polyhistidine-poly(ethylene glycol)diblock copolymer, and an illustrative transfection enhancer comprises a cationic lipid.

Still another illustrative embodiment of the invention comprises a method for increasing efficiency of incorporation of ionic macromolecules into microspheres, the method comprising:
(a) determining the charge of the ionic macromolecules;
(b) forming ionic complexes comprising the ionic macromolecules and a polyelectrolyte-poly(ethylene glycol)diblock copolymer of opposite charge; and
(c) mixing the ionic complexes with a polymer suitable for forming microspheres to form a mixture, and treating the mixture for formation of microspheres. An illustrative polymer suitable for forming microspheres comprises poly(lactide-co-glycolide), and treating the mixture for formation of microspheres can illustratively comprise forming a water-in-oil-in-water emulsion and solvent evaporation.

### BRIEF DESCRIPTION OF THE SEVERAL VIEW OF THE DRAWINGS

FIGS. 1A-B show schematic representations of ionic-complexes between macromolecules and polyelectrolyte-PEG diblock copolymers. FIG. 1A shows complexes comprising negatively charged macromolecules and positively charged polyelectrolyte-PEG diblock copolymers, while FIG. 1B shows complexes comprising positively charge macromolecules and negatively charged polyelectrolyte-PEG diblock copolymers.

FIGS. 2A-B show schematic representations of neutralization of the acidic micro-environment in PLGA microspheres. FIG. 2A shows the presence of ionic species in an acidic microenvironment, and FIG. 2B shows the presence of uncharged species in a neutralized microenvironment.

FIG. 3 shows a schematic representation of coupling chemistry for synthesis of polyelectrolyte-PEG diblock copolymers.

FIGS. 4A-B show particle size as a function of pH with BSA and PEG-PH at 37° C. where PEG-PH concentration was fixed at 0.5 mg/mL (FIG. 4A) and BSA concentration was fixed at 0.5 mg/mL (FIG. 4B): 0.5 mg/mL BSA only (●); 0.5 mg/mL PEG-PH 0.5 only (▪); PEG-PH:BSA=1:3 (∘); PEG-PH:BSA=1:2 (♦); PEG-PH:BSA=1:1 (▴); PEG-PH:BSA=2:1 (▾); PEG-PH:BSA=3:1 (□).

FIG. 5 shows a regression curve for the relation of the ratio of PEG-PH to BSA and particle size at pH 5.5 under a fixed PEG-PH concentration (●, solid line) and under a fixed BSA concentration (◯, dash line).

FIGS. 6A-B show stability of BSA at 37° C.: (FIG. 6A)—the percentage of a-helix structure; (FIG. 6B) the percentage of folding structure; filled symbols show BSA only and hollow symbols show addition of PEG-PH: pH 7.4 (●, ∘); pH 5.5 (▪, □); pH 4.5 (▾, Δ).

FIG. 7 shows the molecular weight change of PLGA microspheres: BSA/PLGA (●); BSA/PH20/PLGA (▪); BSA/PH20-5/PLGA (♦).

FIGS. 8A-L show the morphology of PLGA microspheres at 0 day (FIGS. 8A, C, E, G, I, and K) and 60 days (FIGS. 8B, D, F, H, J, and L), wherein the surface of BSA/PLGA (FIGS. 8A and B), BSA/PH20/PLGA (FIGS. 8C and D); and BSA/PH20-5/PLGA (FIGS. 8E and F) microspheres and the cross-section of BSA/PLGA (FIGS. 8G and H); BSA/PH20/PLGA (FIGS. 8I and J); and BSA/PH20-5/PLGA (FIGS. 8K and L) microspheres are illustrated.

FIGS. 9A-B show in vitro release of BSA from PLGA microspheres at pH 7.4 and 37° C.: (FIG. 9A)—different ratio of PEG-PH to BSA: BSA/PLGA (∘); BSA/PH05/PLGA (♦); BSA/PH10/PLGA (▴); BSA/PH20/PLGA (▪); (FIG. 9B)—different inner aqueous pH: BSA/PLGA (∘); BSA/PH20/PLGA (♦); BSA/PH20-5/PLGA (▴); BSA/PH20-4/PLGA (▪).

FIGS. 10A-B show stability of BSA in PLGA microspheres during in vitro release as measured by the percentage of α-helix structure (FIG. 10A) and the percentage of folding structure (FIG. 10B): BSA/PLGA (∘); BSA/PH20/PLGA (♦); BSA/PH20-5/PLGA (▴); BSA/PH20-4/PLGA (▪).

FIGS. 11A-B show the release profile (FIG. 11A) and stability (FIG. 11B) of insulin from insulin-loaded PLGA microspheres (●), a blend of insulin and poly(L-histidine)-PEG diblock copolymer-loaded microspheres (▪), and a complex of insulin and poly(L-histidine)-PEG diblock copolymer-loaded PLGA microspheres (♦).

FIG. 12A shows the sizes of particles as a function of pH of poly(L-histidine)-PEG diblock copolymer (●), GLP-1 (▪), and a complex of GLP-1 and poly(L-histidine)-PEG diblock copolymer (♦).

FIG. 12B shows the release profile of GLP-1 from GLP-1-loaded PLGA microspheres (●), a blend of GLP-1 and poly(L-histidine)/PEG diblock copolymer loaded PLGA microspheres (▪), and a complex of GLP-1 and poly(L-histidine)-PEG diblock copolymer loaded PLGA microspheres (♦).

FIG. 13 shows complex formation analysis by agarose gel retardation electrophoresis: C— without polymer PLL; lanes 1-9 with 0, 2, 4, 6, 8, 10, 12, 14, 16 μg, respectively, of PEG-poly(L-histidine).

FIG. 14 shows transfection efficiencies in CT 26 cells obtained at different concentrations of PEG-poly(L-histidine): pDNA/PLL (MW 7,000-12,000) is 2:0.4; Lane 1 is the other PLL (MW 22,500) and the PLL/pDNA ration is 2:1.

### DETAILED DESCRIPTION

Before the present compositions and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to an ionic complex containing “a polyelectrolyte-poly(ethylene glycol)diblock copolymer” includes two or more of such polyelectrolyte-poly(ethylene glycol)diblock copolymers, reference to “an ionic complex” includes reference to two or more of such ionic complexes, and reference to “the macromolecule” includes reference to two or more of such macromolecules.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

As used herein, “peptide” means peptides of any length and includes proteins. The terms “polypeptide” and “oligopeptide” may be used herein without any particular intended size limitation, unless a particular size is otherwise stated. Typical of peptides that can be utilized are those selected from group consisting of oxytocin, vasopressin, adrenocorticotrophic hormone, epidermal growth factor, prolactin, luliberin or luteinizing hormone releasing hormone, growth hormone, growth hormone releasing factor, insulin, somatostatin, glucagon, interferon, gastrin, tetragastrin, pentagastrin, urogastroine, secretin, calcitonin, enkephalins, endorphins, angiotensins, renin, bradykinin, bacitracins, polymixins, colistins, tyrocidin, gramicidines, and synthetic analogues, modifications and pharmacologically active fragments thereof, monoclonal antibodies and soluble vaccines. The only limitation to the peptide or protein drug that may be utilized is one of functionality.

As used herein, “PEG-PH” means a polyhistidine-poly(ethylene glycol)diblock copolymer. As used herein, “PEG-PH:BSA” means an ionic complex of a polyhistidine-poly(ethylene glycol)diblock copolymer and bovine serum albumin.

As used herein, the term “effective amount” refers to the quantity of a component that is sufficient to yield a desired therapeutic response without undue adverse effects (such as toxicity, irritation, or allergic response) commensurate with a reasonable benefit/risk ratio when used in the manner of this invention. The specific “effective amount” will, obviously, vary with such factors as the particular condition that is being treated, the severity of the condition, the duration of the treatment, the physical condition of the patient, the nature of concurrent therapy (if any), and the specific formulation used in the present invention.

As used herein, “administering” and similar terms mean delivering the composition to the individual being treated such that the composition is capable of being circulated systemically to the parts of the body where the pharmaceutical portion of the composition exert its therapeutic effect. Thus, the composition is preferably administered to the individual by systemic administration, typically by subcutaneous, intramuscular, or intravenous administration, or intraperitoneal administration. Injectables for such use can be prepared in conventional forms, either as a liquid solution or suspension or in a solid form suitable for preparation as a solution or suspension in a liquid prior to injection, or as an emulsion. Suitable excipients include, for example, water, saline, dextrose, glycerol, ethanol, and the like; and if desired, minor amounts of auxiliary substances such as wetting or emulsifying agents, buffers, and the like can be added.

This invention relates to stabilization, storage, and delivery of biologically active molecules, such as proteins, peptides, nucleic acids, plasmids, and antisense oligonucleotides (ODNs). In particular this invention relates to stabilization of biological macromolecules during the harsh processing conditions commonly used in manufacture of pharmaceutical formulations.

The present invention is further directed to encapsulation of such biological macromolecules into compositions or formulations for biological delivery for human and veterinary use. The successful encapsulation in a formulation comprising a polymeric carrier matrix helps in preserving the native biologically active tertiary structure of the macromolecule and create a reservoir that can slowly release active macromolecules. Such polymeric carriers generally include biocompatible and biodegradable polymers, such as poly(lactic-co-glycolic acid) (PLGA).

The present invention is further aimed at controlling the spatial and temporal characteristics of the release pattern of biological macromolecules. The biologically active molecule is released in a controlled manner over a period of time as influenced by encapsulation technique, polymer composition, and formulation conditions used.

The invention is based, in part, on observations that adding a polycationic condensing agent to a polyanionic bioactive agent during the production of microspheres increases the efficiency with which the bioactive agent is incorporated into the microspheres. Efforts to formulate protein and/or DNA within microspheres are hampered by several problems. For example, present methods exhibit very low efficiencies of incorporation. Most of the protein and/or DNA present in the emulsion used to prepare the microspheres does not get into the finished microspheres. Methods that enhance the efficiency of incorporation of protein and/or DNA would have the beneficial effect of requiring less protein and/or DNA to produce an end product with a given amount of incorporated protein and/or DNA. Such methods may also increase the amount of protein and/or DNA incorporated into each microsphere, allowing the introduction of fewer microspheres into the treatment site to deliver a given amount of total protein and/or DNA to a patient. Moreover, incorporation of protein and/or DNA into microspheres is impaired by fragmentation of the protein and/or DNA. In one common method, protein and/or DNA microspheres are formed using a water-in-oil-in-water (W/O/W) double emulsion method. Each of the two emulsifying steps frequently involves sonication, which causes fragmentation of the protein and/or DNA. Having available methods for increasing the efficiency of incorporating DNA within microspheres without inducing protein and/or DNA fragmentation would be extremely advantageous. Such protein/DNA-containing microspheres would facilitate intracellular as well as extracellular controlled or sustained release of therapeutic pharmaceuticals at the site of medical intervention.

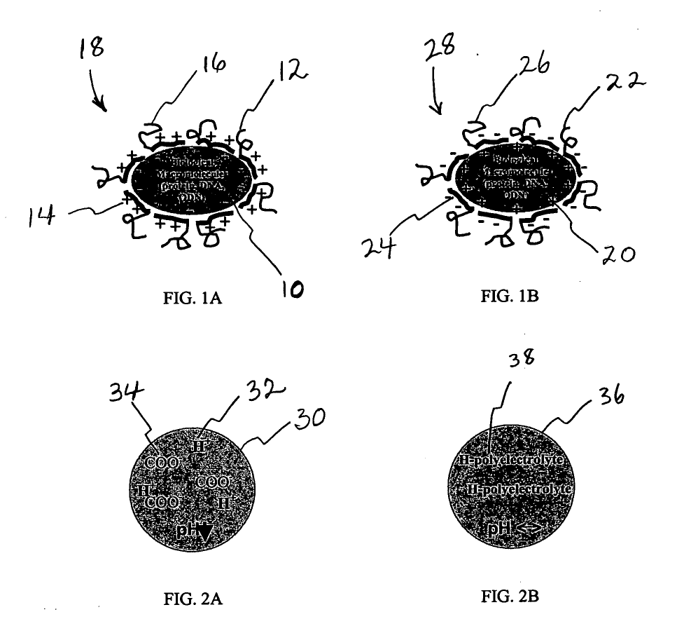

To retain the stability of biological macromolecules, a first illustrative strategy involves increasing the hydrophilicity of biological macromolecules by “physical PEGylation” using a polyelectrolyte. PEGylation as known today is the covalent attachment of PEG to modify the properties of biological macromolecules. “Physical PEGylation” is inducement of interactions by ionic complex formation between biological macromolecules and polyelectrolyte-PEG diblock copolymers. In a specific pH range, the polyelectrolyte has the charge opposite to the biological macromolecule. Therefore, the polyelectrolyte-PEG diblock copolymer and biological macromolecules form an ionic complex as schematically presented in FIGS. 1A-B. “Physical PEGylation” increases the hydrophilicity of biological macromolecules, which reduces their aggregation. It decreases, and ideally removes, the loss of biological activity that correlates with the PEG attachment site.

FIG. 1A shows a polyanionic macromolecule 10 bearing negative charges. A polycationic polyelectrolyte-PEG diblock copolymer 12, comprised of a polycationic polyelectrolyte block 14 and a PEG block 16, ionically bonds to the polyanionic macromolecule 10, resulting in an ionic complex 18 in which the polyanionic macromolecule 10 and the polycationic polyelectrolyte block 14 bind to each other by ionic bonds, and the PEG block 16 remains unbound. Similarly, FIG. 1B shows a polycationic macromolecule 20 bearing positive charges. A polyanionic polyelectrolyte-PEG diblock copolymer 22, comprised of a polyanionic polyelectrolyte block 24 and a PEG block 26, ionically bonds to the polycationic macromolecule 20, resulting in an ionic complex 28 in which the polycationic macromolecule 20 and the polyanionic polyelectrolyte block 24 bind to each other by ionic bonds, and the PEG block 26 remains unbound.

A second illustrative strategy is neutralization of the acidic micro-environment in PLGA microspheres. Modification of the formulation of PLGA microspheres is one of the methods for retaining the stability of the biological macromolecules residing in the PLGA microspheres during administration and for controlling their release. The PLGA degradation process is an acid-catalyzed hydrolysis that produces carboxylic acids. To neutralize the acidic micro-environment, polyelectrolytes, which are known to have a proton sponge effect, are provided such that they help in neutralization of the acidic conditions in the interior of PLGA microspheres.

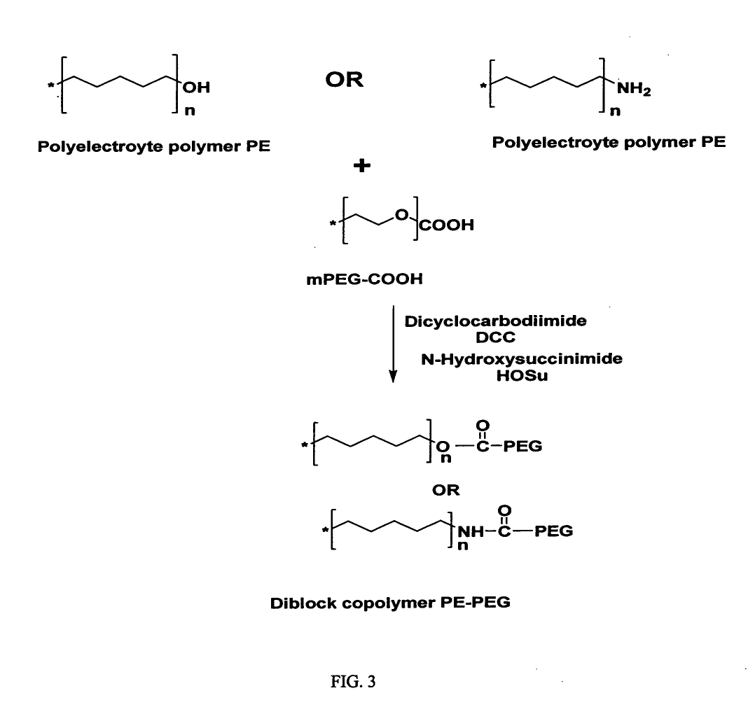

FIGS. 2A-B show a schematic representation of this second illustrative strategy. FIG. 2A shows a PLGA microsphere 30 with hydrogen ions 32 and ionized carboxylic acid groups 34 that are produced during hydrolysis of PLGA. FIG. 2B shows an improved PLGA microsphere 36 according to the present invention wherein polyelectrolyte moieties 38 are present to neutralize the hydrogen ions produced during hydrolysis of PLGA.

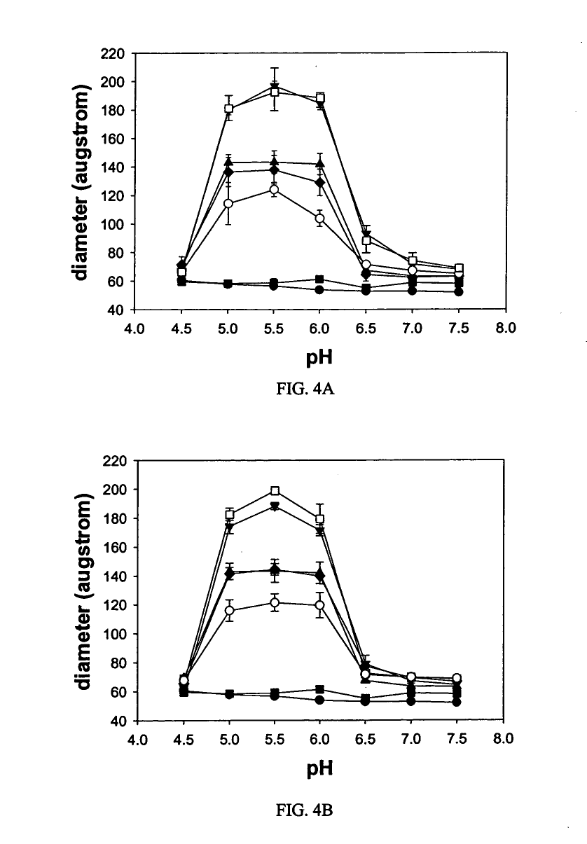

Polyelectrolyte-polyethylene glycol diblock copolymers (PE-PEG) used in the present invention can be synthesized by conjugating a polyelectrolyte block to a PEG block, as is schematically illustrated in FIG. 3. Briefly, this scheme involves activating a monocarboxylic acid derivative of PEG, then reacting the polyelectrolyte with the activated PEG, thus resulting in the diblock copolymer. An illustrative method of synthesizing such copolymers is described in U.S. patent application Ser. No. 10/640,739, and E. S. Lee et al., Polymeric micelle for tumor pH and folate mediated targeting, 91 J. Control. Release 103-113 (2003), both of which are hereby incorporated by reference. PEG is commercially available from numerous sources. Illustratively, the PEG has a molecular weight in the range of about 1000-10,000 Da (1-10 kDa).

Polyelectrolytes that can be used according to the present invention include, without being limited to these illustrative examples, poly(histidine), poly(lysine), poly(arginine), poly(acrylic acid), poly(methacrylic acid), poly(glutamic acid), poly(aspartic acid), sulfonamide-based pH sensitive polymers such as vinyl-sulfonamide oligomers and poly(glutamic acid-sulfonamide), copolymers of polyhistidine with poly(phenylalanine) or poly(leucine) or poly(isoleucine), poly(diallyldimethylammonium chloride) (PDADMAC), sodium poly(2-acrylamide-2-methylpropane sulfonate) (PAMPS), copolymer of (2-acrylamide-2-methylpropane sulfonate) with N-vinylpyrrolidone (NVP-co-AMPS), polybrene(1,5-dimethyl-1,5-diazaundecamethylene polymethobromide), polyethylenimine and its derivatives(PEI), sodium poly(styrene sulfonate), poly(allylamine hydrochloride), sulfonated poly(glutamic acid), poly(vinyl alcohol sulfonate) potassium salt, hyaluronis acid sodium salt, sodium alginate, poly(2-methacryloyloxyethyl dihydrogen phosphate-co-N-isopropylacrylamide), poly(N-ethyl-4-vinyl-pyridinium bromide), poly(N-isopropyl-acrylamide-co-acrylic acid), and the like.

Macromolecules that can be stabilized and delivered according to the present invention include proteins and peptides, such as enzymes, hormones, antigens and vaccines; and nucleic acids, such as plasmids and antisense oligonucleotides (ODNs). Illustrative proteins and peptides include salmon calcitonin (sCT), insulin, glucagon-like peptide (GLP), erythropoietin (EPO), thrombopoietin (TPO), epidermal growth factor, vascular endothelial growth factor (VEGF), platelet-derived growth factor (PDGF), interferons (IFN-α, IFN-β, IFN-γ, IFN-τ), interleukins (e.g., IL-1 through IL-1 5), colony-stimulating factors (G-CSF, M-CSF, GM-CSF), tumor necrosis factors (TNF-α, TNF-β), fibroblast growth factor (FGF), and neurotrophic factors, and the like. Illustrative enzymes include adenosine deaminase, asparaginase, tissue plasminogen activator (TPA), urokinase and streptokinase, superoxide dismutase, and digestive enzymes, and the like. Illustrative hormones include human growth hormone (HGH), luteinizing hormone (LH), and gonadotropin peptides, and the like. Illustrative antigens and vaccines include tetanus vaccine, diphtheria vaccine, pertussis vaccine, and influenza vaccine, and the like.

Ionic complexes of the polyelectrolyte-PEG diblock copolymers and the ionic macromolecules can be stored according to methods well known in the art. Similarly, microspheres containing such ionic complexes can be stored and used according to methods well known in the art, taking into consideration the particular macromolecules that are being used.

### EXAMPLE 1

Bovine Serum Albumin (BSA) and Poly(L-Histidine)-PEG Diblock Copolymer

The isoelectric point of BSA is about pH 4.9 and the pK_{b} value of poly(L-histidine) is about pH 7.0. Therefore, BSA and poly(L-histidine)-PEG diblock copolymer make an ionic complex in the pH range of about 4.9 to 7.0. These complexes aid in retention of stability of BSA. Additionally and more significantly, the release profile of BSA from PLGA microspheres undergoes a change of pattern and extent, as set out below.

Materials. Poly(DL-lactide-co-glycolide) (PLGA, copolymer composition 50:50; MW 40,000˜75,000), bovine serum albumin (BSA, fraction V), and poly(vinyl alcohol) (PVA, Mw 85,000˜146,000) were purchased from Aldrich Chemical Co. Methylene chloride (MC) from Fisher Scientific Co. was used without further purification. Poly(L-histidine)-poly(ethylene glycol) (PH-PEG) diblock copolymers were synthesized according to the method of E. S. Lee et al., supra.

Particle Size and Stability of BSA. The particle size of PH-PEG and BSA particles were was measured at various pH values, from 4.5 to 7.5, using a Zeta Sizer (Brookhaven Instruments Corp. ZetaPALS). PH-PEG concentration was fixed at 0.5 mg/mL. BSA concentration varied from 0.17 to 1.5 mg/mL. The weight ratios of PH-PEG to BSA were 0.33 to 3.

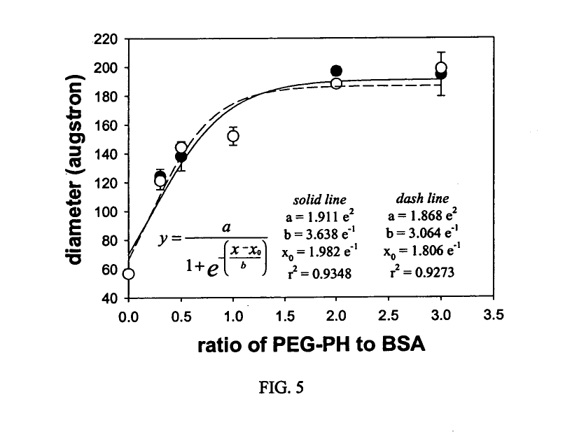

The size of PEG-PH and BSA particles as a function of concentration was plotted versus pH, as shown in FIGS. 4A-B. At pH 5.0 to 6.0, the size dramatically increased compared with other pH values. Both BSA and PEG-PH bear positive charges below pH 4.9, because the isoelectric point of BSA is pH 4.9 and the pK_{b} of PEG-PH is 7.0. Therefore, BSA and PEG-PH could not make a complex by ionic interactions below that pH, since BSA and PEG-PH would repel each other. Similarly, BSA and PEG-PH could not make a complex above pH 7.0, because BSA bears a negative charge and PEG-PH is neutral. However, they could form a complex in the range of pH 4.9 to 7.0 because BSA has a negative charge and PEG-PH has a positive charge. Electrostatic attraction led to formation of an ionic complex.

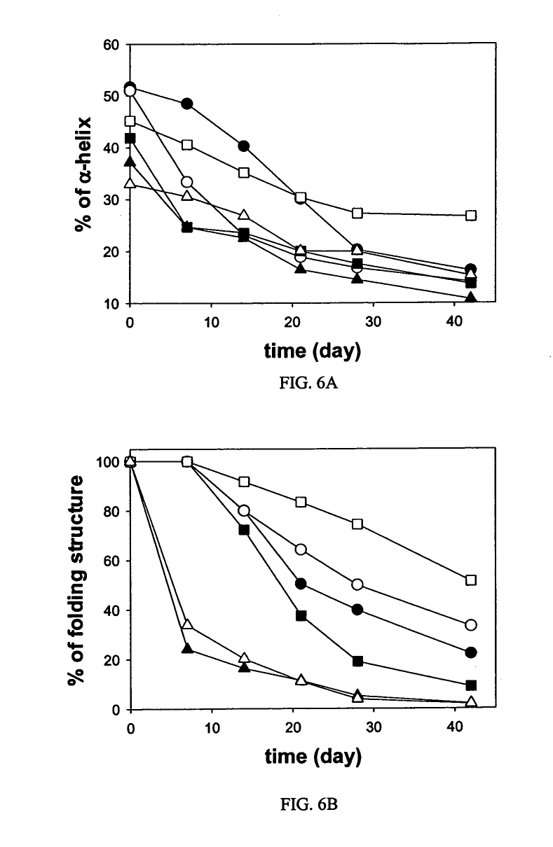

FIG. 5 shows the relation of particle size to the ratio of PEG-PH concentration to BSA concentration. As the ratio increased, the particle size also increased. Both in the case of fixed PEG-PH concentration (0.5 mg/mL) and fixed BSA concentration (0.5 mg/mL), particle size appeared to level off after the ratio reached 2.0. It can be inferred that BSA and PEG-PH completely complexed at that ratio. The molar ratio corresponding to this weight ratio is about 18.8. Thus, on average a BSA molecule and about 18.8 PEG-PH molecules formed an ionic complex.

Stability was monitored using circular dichroism (CD). The CD spectra were recorded on a Jasco J-720 spectropolarimeter. A quartz cuvette of 0.1 cm path length was used. The spectra were scanned between 190 and 260 nm with 0.5 nm resolution. Sixteen (16) scans were accumulated with a scan rate of 100 nm min⁻¹ and a time constant of 0.125 s. The spectral analysis was performed by deconvolution of CD spectra. The measured spectra were deconvoluted with CDNN freeware (version 2.1). To analyze the folding structure of BSA, the emission peak of tryptophan in BSA was measured by fluorescence (PerkinElmer LS55). This peak in the folded structure was 335 nm, and the peak in the unfolded structure was in the range of 355 to 380 nm.

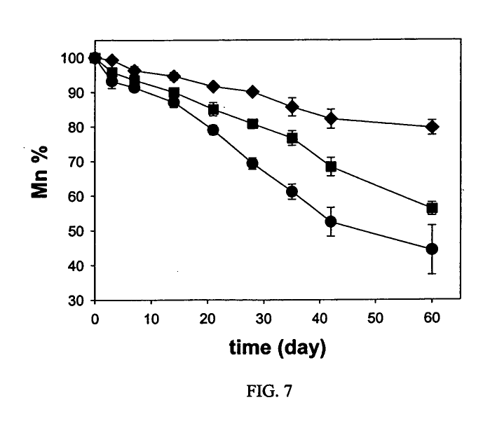

As seen in FIGS. 6A-B, there is a large and dramatic improvement in maintenance of stability of BSA upon addition of PEG-PH at pH 5.5 as compared the absence of PEG-PH. There were no improvements or differences in stability at pH 4.5 and pH 7.5. With respect to secondary structure, the percentage of a-helix of BSA was maintained at 30 % following addition of PEG-PH at pH 5.5, while it decreased to 15% after 42 days in the absence of PEG-PH. The percentage of a-helix of native BSA was about 50%. Therefore, about 60% of BSA molecules maintained the native a-helix structure upon addition of PEG-PH at pH 5.5. Similarly, as seen with respect to tertiary structure, the percentage of folding structure of BSA was maintained at 60% under the same conditions. Therefore, BSA and PEG-PH formed an ionic complex at pH 5.5. The poly(L-histidine) block of PEG-PH was bonded to the BSA surface by ionic bonds, and the PEG block covered the BSA surface. Therefore, the BSA surface was changed to highly hydrophilic because of the presence of PEG.

Preparation of Microspheres. PLGA microspheres were prepared by the conventional W/O/W emulsion and solvent evaporation technique. First, BSA and PH-PEG were dissolved in purified water. This solution serving as an internal aqueous phase was emulsified with methylene chloride containing PLGA for 1 minute using a homogenizer. This W/O emulsion was poured into poly(vinyl alcohol) (PVA) and sodium chloride (NaCl) solution as the external aqueous phase. Emulsification was continued using a mechanical stirrer at 2000 rpm for 1 minute. This dispersion was stirred for 4 hrs at 35° C. for solvent evaporation. The microspheres were collected by centrifugation at 3000 rpm for 10 minutes. The microspheres were washed with water and freeze dried for at least 24 hrs.

PLGA microspheres containing only BSA (BSA/PLGA) were smooth and spherical with a mean size of 23.4±2.3 nm. The loading efficiency of BSA was 81.3±7.4% as determined by UV spectrophotometry. The characteristics of other microspheres that were prepared under other conditions was similar to those of PLGA microspheres containing only BSA. Characteristics including mean size and loading efficiency of all microspheres was summarized in Table 1. The mean size and loading efficiency of BSA into the microspheres were very similar regardless of addition of PEG-PH.

TABLE 1
The Characteristics of PLGA Microspheres

| | | | | Loading |
|---|---|---|---|---|
| | Ratio | | Size (μm) | efficiency (%) |
| | BSA | PEG-PH | mean ± S.D. | mean ± S.D. |
| BSA/PLGA | 1 | 0^{a} | 23.4 ± 2.3 | 81.3 ± 7.4 |
| BSA/PH05/PLGA | 1 | 0.5^{a} | 27.4 ± 2.5 | 84.1 ± 8.1 |
| BSA/PH10/PLGA | 1 | 1^{a} | 26.7 ± 2.7 | 86.4 ± 4.3 |
| BSA/PH20/PLGA | 1 | 2^{a} | 28.8 ± 4.2 | 87.3 ± 8.4 |
| BSA/PH20-5/PLGA | 1 | 2^{b} | 34.3 ± 3.9 | 88.1 ± 7.2 |
| BSA/PH20-4/PLGA | 1 | 2^{c} | 29.4 ± 5.4 | 86.3 ± 6.9 |

^{a}pH 7.4
^{b}pH 5.5
^{c}pH 4.5

In vitro Degradation of PLGA Microspheres. Degradation studies were accomplished in phosphate buffer saline (PBS) (pH 7.4) solution. The microspheres were immersed in vials containing 20 mL of PBS solution. Each sample was periodically removed and then dried in vacuo for 24 hrs before being measured. Until analysis, samples were kept in a desiccator. The change of molecular weight was obtained by GPC using N,N-dimethylformamide (DMF) as an eluent. It was determined by comparing the molecular weight at a given time (Mnₜ) with the initial molecular weight (Mn₀). The change of molecular weight was defined as:
Mn%=(Mnₜ/Mn₀)×100

The surface morphology and inner structure of microspheres were investigated by scanning electron microscope (SEM, Hitachi S-3000N). To assess the surface morphology, microspheres were mounted onto metal stubs using double-sided adhesive tape, vacuum-coated with a gold film and directly analyzed by SEM. To study the interior structure, microspheres embedded in a gelatin and cross-sectioned using an ultra-microtome was coated with gold and viewed by SEM.

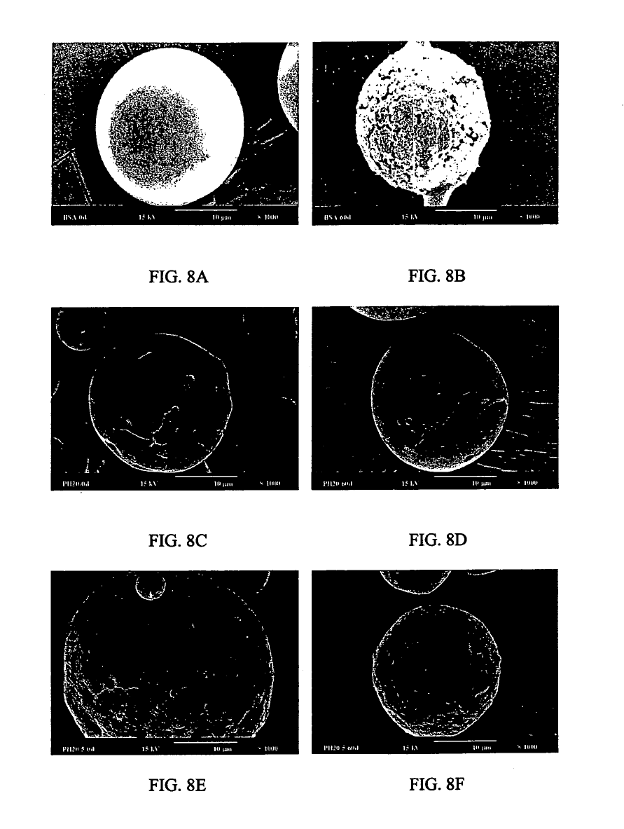

FIG. 7 shows change in molecular weight of PLGA microspheres that were prepared under different conditions. One was plain PLGA microspheres, in which PEG-PH and BSA were dissolved in pH 7.4 PBS and loaded into PLGA microspheres (BSA/PH20/PLGA). In the other, PEG-PH and BSA were dissolved in pH 5.5 PBS and loaded into PLGA microspheres (BSA/PH20-5/PLGA). After 60 days, the molecular weight decreased to 50% in PLGA microspheres, while it decreased only 10% in BSA/PH20-5/PLGA microspheres.

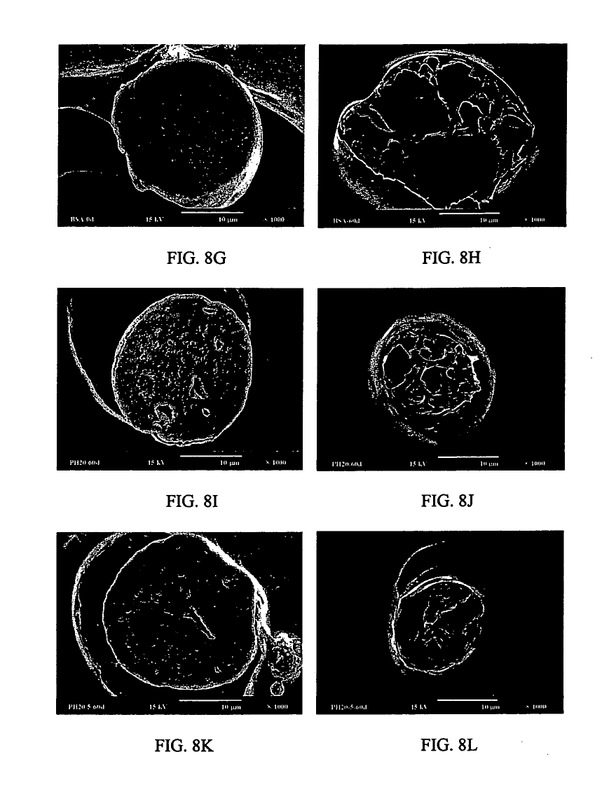

The surface and cross section morphology of PLGA microspheres are presented in FIGS. 8A-L. Before the degradation test, surface morphology was smooth and the cross section was a dense structure, regardless of the pH at which the microspheres were prepared. However, these were very different from each other after 60 days. In PLGA microspheres, many pores and distinct hollow cores were observed on the surface and in cross sections, while a few pores were observed on the surface, but small holes existed in cross sections of BSA/PH20/PLGA microspheres. On the other hand, a few pores and cracks were observed on the surface and in cross sections of BSA/PH20-5/PLGA microspheres. From these results, namely molecular weigh change and surface and cross sectional morphology of PLGA microspheres, it can be inferred that PEG-PH reduced degradation of PLGA microspheres. PEG-PH neutralized the acidic microenvironment within PLGA microspheres and reduced the possibility of acid-catalyzed hydrolysis.

In vitro Release and Stability of BSA. BSA release was monitored in PBS. Microspheres were immersed in release medium and incubated under mild stirring at 37° C. The samples were taken at various time points after suspension was centrifuged. Protein content in release samples was determined by UV spectrophotometry. The stability of BSA was analyzed as previously described in the section “Determination of Particle Size and Stability of BSA.”

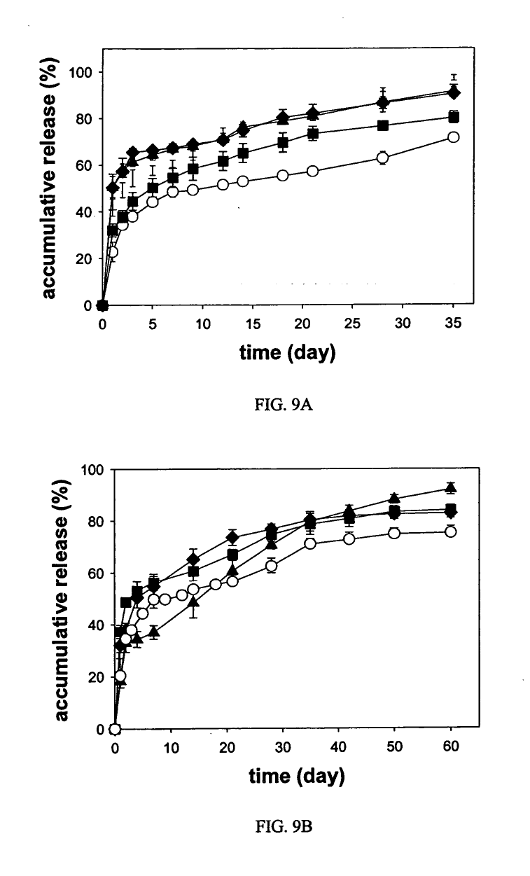

Release profiles of BSA from PLGA microspheres were evaluated at different ratios of PEG-PH to BSA and are shown in FIG. 9A. All profiles are similar except for the initial burst release within 2 days. After the burst release, BSA release from PLGA microspheres slowed. PEG-PH had no effect on the release profiles of BSA, because it did not make any ionic complex with BSA at pH 7.4. However, the release profile of BSA from BSA/PH20-5/PLGA microspheres, as shown in FIG. 9B, was different. Compared to BSA/PLGA microspheres, BSA release was much slower initially and later showed a linear release profile for up to 8 weeks. The release from BSA/PLGA microspheres almost stopped at a 70% level. The BSA complexed with PEG-PH was more hydrophilic than without PEG-PH or when physically blended with PEG-PH. Therefore, BSA was predominantly located, not on the surface of microspheres, but in the core of microspheres. It reduced the initial burst release, which was due to fast release of BSA from the surface of PLGA microspheres.

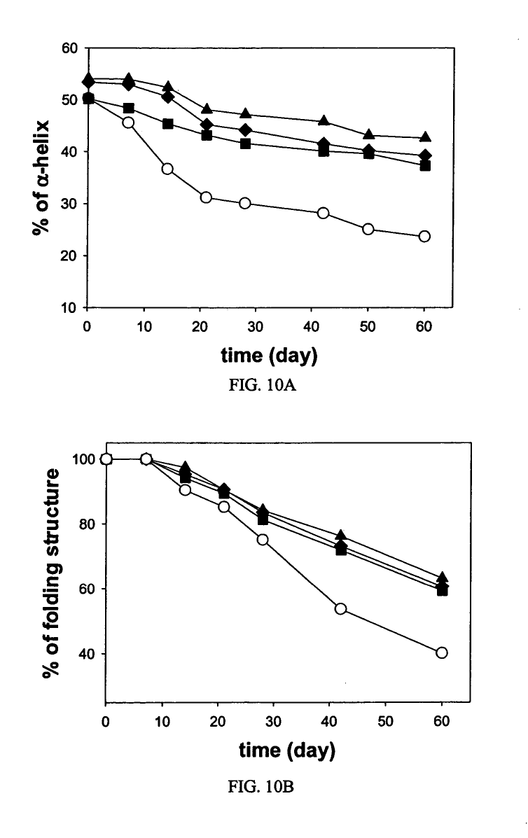

FIGS. 10A-B show the stability of BSA in various PLGA microspheres during an in vitro release test. Stability, as judged by the secondary and tertiary structure of BSA, dramatically increased on addition of PEG-PH. However, there were no sharp differences in stability profiles among the microspheres prepared under different pH conditions (BSA/PH20/PLGA, BSA/PH20-5/PLGA, and BSA/PH20-4/PLGA). It can be inferred that PEG-PH was effective in maintaining stability of BSA in PLGA microspheres regardless of whether PEG-PH was complexed with BSA or merely mixed with BSA. PEG-PH prevented the aggregation of BSA since PEG-PH covers the surface of BSA, leading to reduced hydrophobic interactions that are responsible for aggregation.

### EXAMPLE 2

Insulin and Poly(L-Histidine)-PEG Diblock Copolymer

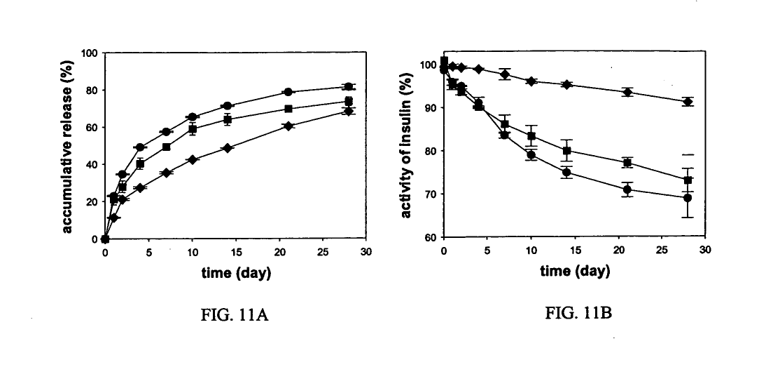

The isoelectric point of insulin is about pH 5.4 and the pKb value of poly(L-histidine) is about pH 7.0. Therefore, insulin and poly(L-histidine)-PEG diblock copolymer make an ionic complex in the pH range of about 5.4 to 7.0. FIG. 11A shows the release profile of insulin from PLGA microspheres, and FIG. 11B shows the stability that was measured by radioimmunoassay (RIA) of released insulin.

Ionic complexes of insulin and poly(L-histidine)-PEG diblock copolymer were made and incorporated into microspheres according to the following method. Poly(L-histidine)-PEG diblock copolymer was synthesized according to the method of U.S. patent application Ser. No. 10/640,739, and E. S. Lee et al., Polymeric micelle for tumor pH and folate mediated targeting, 91 J. Control. Release 103-113 (2003). Insulin and poly(L-histidine)-PEG diblock copolymer were dissolved in pH 6.0 buffer solution and then poured into a methylene chloride solution of PLGA. Microspheres were prepared as a traditional W/O/W emulsion, according to methods well known in the art. Control blends of insulin and poly(L-histidine)-PEG diblock copolymer were made according to similar methods, except the pH of the aqueous buffer was pH 7.4. Under these conditions, insulin and poly(L-histidine)-PEG diblock copolymer did not make ionic complexes. FIGS. 11A-B demonstrate that the formation of the complex dramatically improved the release profile and stability of insulin as compared to a mere blend of the ingredients.

### EXAMPLE 3

Glucagon Like Peptide-1 (GLP-1) and Poly(L-Histidine)-PEG Diblock Copolymer

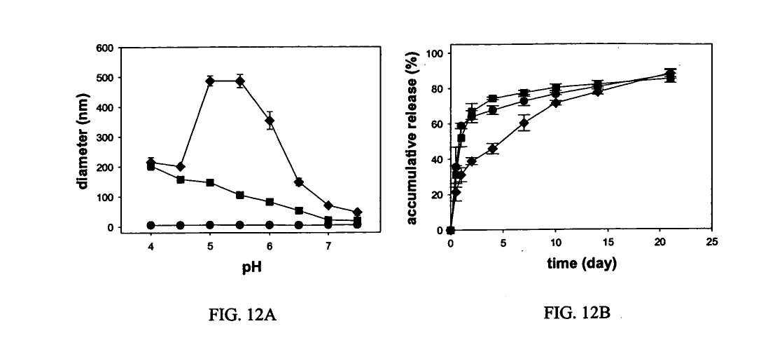

The isoelectric point of GLP-1 is about 4.6. Therefore, GLP-1 makes an ionic complex with poly(L-histidine)-PEG diblock copolymer in the pH range of about 4.6 to 7.0. FIG. 12A shows that the size of GLP-1 and poly(L-histidine)-PEG diblock copolymer increased in that pH range. GLP-1 and poly(L-histidine)-PEG diblock copolymer loaded PLGA microspheres were prepared according to the method of Example 2. A blend of GLP-1-loaded microspheres did not show any improvement for controlled release, while a complex of GLP-1 and poly(L-histidine)-PEG diblock copolymer-loaded microspheres reduced the burst release and showed controlled release similar to zero order release pattern (FIG. 12B).

### EXAMPLE 4

Gene Delivery Using Polyelectrolyte (Polyethylene Glycol-Polyhistidine: PEG-PH)

Preparation of polymer/DNA complex. The polymer-DNA complex formation was initiated by the process of self-assembly. First, poly(L-lysine) (PLL) solution (0.4 μg) was added into DNA solution (2.0 μg) and left undisturbed for 30 minutes (DNA/PLL weight ratio 5:1). Subsequently, polyethylene glycol-polyhistidine (PEG-PH) solution was added in varying amounts (2-16 μg) into the PLL/DNA solution. Complex formation was monitored by routine 1% agarose gel electrophoresis.

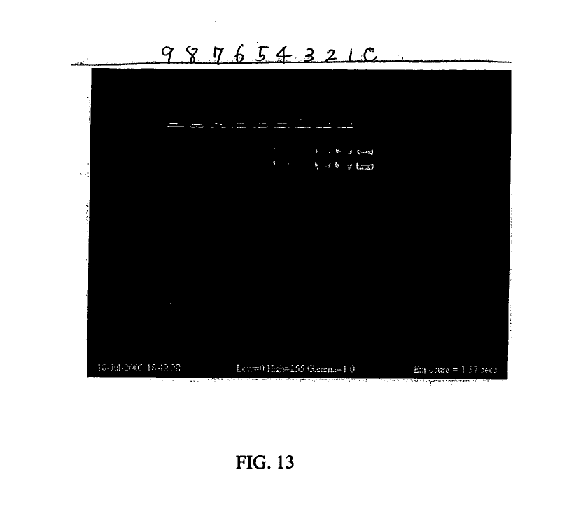

A gel retardation study was carried out in order to confirm self-assembling complexes of PLL/PEG-poly(His)/DNA (FIG. 13). Increases in the amount of PEG-poly(His) leads to different complexation patterns identified by 1.0% agarose gel retardation assay. These results showed that PEG-Poly(His) was able to condense plasmid DNA into small particles above 8.0 μg (weight ratio DNA/PEG-poly(His) 1:4). The average effective diameter of the complex was 200 nm as determined by light scattering.

In vitro transfection. Colon carcinoma CT26 cells were cultured on 24-well plates at a concentration of 2×10⁴ cells per well for 24 h. Cultures were initiated in DMEM medium containing 10% fetal bovine serum, supplemented with 100 Uml penicillin at 37° C. for 24 h under 5% carbon dioxide atmosphere. The growth medium was removed, and cells were washed with phosphate-buffered saline (PBS) pH 7.4. Following addition of the transfection medium, CT26 cells were incubated for 24 h. The medium was collected and stored at −70° C. until analysis for mIL-10 expression assay.

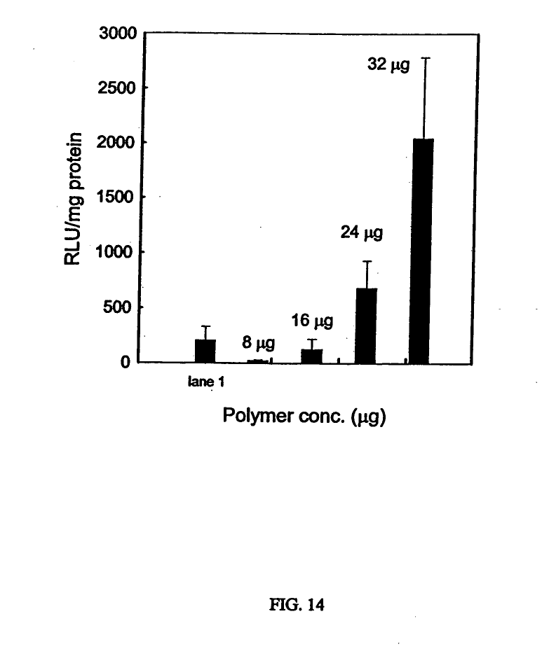

This was carried out to test whether PEG-poly(His) had an enhanced effect on plasmid delivery into CT26 cells. The efficiency of PEG-poly(His) mediated-transfection to CT26 cells was significantly higher than without PEG-poly(His). Moreover, the transfection efficiency was 10 times greater than that of PLL (MW: 22,500 Da, weight ratio of DNA/PLL 1:2), which is known to have highest transfection efficiency (FIG. 14).

1. A composition comprising an ionic complex of (a) a biological macromolecule having a charge, and (b) a polyelectrolyte-poly(ethylene glycol)diblock copolymer having an opposite charge.

2. The composition of claim 1 wherein the biological macromolecule is a peptide.

3. The composition of claim 2 wherein the peptide is an enzyme, hormone, antigen, vaccine, or mixture thereof.

4. The composition of claim 1 wherein the biological macromolecule is a nucleic acid.

5. The composition of claim 4 wherein the nucleic acid is a plasmid.

6. The composition of claim 4 wherein the nucleic acid is an antisense oligonucleotide.

7. The composition of claim 1 wherein the polyelectrolyte-poly(ethylene glycol)diblock copolymer comprises a polycation-poly(ethylene glycol)diblock copolymer.

8. The composition of claim 1 wherein the polyelectrolyte-poly(ethylene glycol)diblock copolymer comprises a polyanion-poly(ethylene glycol)diblock copolymer.

9. The composition of claim 1 wherein the polyelectrolyte-poly(ethylene glycol)diblock copolymer comprises a poly(L-histidine)-poly(ethylene glycol)diblock copolymer.

10. The composition of claim 1 wherein the polyelectrolyte-poly(ethylene glycol)diblock copolymer comprises a polyelectrolyte block selected from the group consisting of poly(histidine), poly(lysine), poly(arginine), poly(acrylic acid), poly(methacrylic acid), poly(glutamic acid), poly(aspartic acid), vinyl-sulfonamide oligomers, poly(glutamic acid-sulfonamide), poly(histidine)-poly(phenylalanine)copolymers, poly(histidine)-poly(leucine)copolymers, poly(histidine)-poly(isoleucine)copolymers, poly(diallyldimethylammonium chloride), sodium poly(2-acrylamide-2-methylpropane sulfonate), copolymer of (2-acrylamide-2-methylpropane sulfonate) with -vinylpyrrolidone (NVP-co-AMPS), polybrene(1,5-dimethyl-1,5-diazaundecamethylene polymethobromide), polyethylenimine and its derivatives, sodium poly(styrene sulfonate), poly(allylamine hydrochloride), sulfonated poly(glutamic acid), poly(vinyl alcohol sulfonate)potassium salt, hyaluronis acid sodium salt, sodium alginate, poly(2-methacryloyloxyethyl dihydrogen phosphate-co- -isopropylacrylamide), poly(N-ethyl-4-vinyl-pyridinium bromide), and poly(N-isopropyl-acrylamide-co-acrylic acid).

11. A composition comprising a microsphere encapsulating an ionic complex of (a) a biological macromolecule having a charge, and (b) a polyelectrolyte-poly(ethylene glycol)diblock copolymer having an opposite charge.

12. The composition of claim 11 wherein the microsphere comprises a biocompatible polymer.

13. The composition of claim 12 wherein the biocompatible polymer comprises poly(lactide-co-glycolide).

14. The composition of claim 11 wherein the biological macromolecule is a peptide.

15. The composition of claim 14 wherein the peptide is an enzyme, hormone, antigen, vaccine, or mixture thereof.

16. The composition of claim 11 wherein the biological macromolecule is a nucleic acid.

17. The composition of claim 16 wherein the nucleic acid is a plasmid.

18. The composition of claim 16 wherein the nucleic acid is an antisense oligonucleotide.

19. The composition of claim 11 wherein the polyelectrolyte-poly(ethylene glycol)diblock copolymer comprises a polycation-poly(ethylene glycol)diblock copolymer.

20. The composition of claim 11 wherein the polyelectrolyte-poly(ethylene glycol)diblock copolymer comprises a polyanion-poly(ethylene glycol)diblock copolymer.

21. The composition of claim 11 wherein the polyelectrolyte-poly(ethylene glycol)diblock copolymer comprises a poly(L-histidine)-poly(ethylene glycol)diblock copolymer.

22. The composition of claim 11 wherein the polyelectrolyte-poly(ethylene glycol)diblock copolymer comprises a polyelectrolyte block selected from the group consisting of poly(histidine), poly(lysine), poly(arginine), poly(acrylic acid), poly(methacrylic acid), poly(glutamic acid), poly(aspartic acid), vinyl-sulfonamide oligomers, poly(glutamic acid-sulfonamide), poly(histidine)-poly(phenylalanine)copolymers, poly(histidine)-poly(leucine)copolymers, poly(histidine)-poly(isoleucine)copolymers, poly(diallyldimethylammonium chloride), sodium poly(2-acrylamide-2-methylpropane sulfonate), copolymer of (2-acrylamide-2-methylpropane sulfonate) with -vinylpyrrolidone (NVP-co-AMPS), polybrene(1,5-dimethyl-1,5-diazaundecamethylene polymethobromide), polyethylenimine and its derivatives, sodium poly(styrene sulfonate), poly(allylamine hydrochloride), sulfonated poly(glutamic acid), poly(vinyl alcohol sulfonate) potassium salt, hyaluronis acid sodium salt, sodium alginate, poly(2-methacryloyloxyethyl dihydrogen phosphate-co- -isopropylacrylamide), poly(N-ethyl-4-vinyl-pyridinium bromide), and poly(N-isopropyl-acrylamide-co-acrylic acid).

23. A method of stabilizing an ionic macromolecule against inactivation comprises:
(a) determining the charge of the ionic macromolecule; and
(b) complexing the ionic macromolecule with a polyelectrolyte-poly(ethylene glycol)diblock copolymer of opposite charge.

24. The method of claim 23 wherein the biological macromolecule is a peptide.

25. The method of claim 24 wherein the peptide is an enzyme, hormone, antigen, vaccine, or mixture thereof.

26. The method of claim 23 wherein the biological macromolecule is a nucleic acid.

27. The method of claim 26 wherein the nucleic acid is a plasmid.

28. The method of claim 26 wherein the nucleic acid is an antisense oligonucleotide.

29. The method of claim 23 wherein the polyelectrolyte-poly(ethylene glycol)diblock copolymer comprises a polycation-poly(ethylene glycol)diblock copolymer.

30. The method of claim 23 wherein the polyelectrolyte-poly(ethylene glycol)diblock copolymer comprises a polyanion-poly(ethylene glycol)diblock copolymer.

31. The method of claim 23 wherein the polyelectrolyte-poly(ethylene glycol)diblock copolymer comprises a poly(L-histidine)-poly(ethylene glycol)diblock copolymer.

32. The composition of claim 23 wherein the polyelectrolyte-poly(ethylene glycol)diblock copolymer comprises a polyelectrolyte block selected from the group consisting of poly(histidine), poly(lysine), poly(arginine), poly(acrylic acid), poly(methacrylic acid), poly(glutamic acid), poly(aspartic acid), vinyl-sulfonamide oligomers, poly(glutamic acid-sulfonamide), poly(histidine)-poly(phenylalanine)copolymers, poly(histidine)-poly(leucine)copolymers, poly(histidine)-poly(isoleucine)copolymers, poly(diallyldimethylammonium chloride), sodium poly(2-acrylamide-2-methylpropane sulfonate), copolymer of (2-acrylamide-2-methylpropane sulfonate) with -vinylpyrrolidone (NVP-co-AMPS), polybrene(1,5-dimethyl-1,5-diazaundecamethylene polymethobromide), polyethylenimine and its derivatives, sodium poly(styrene sulfonate), poly(allylamine hydrochloride), sulfonated poly(glutamic acid), poly(vinyl alcohol sulfonate) potassium salt, hyaluronis acid sodium salt, sodium alginate, poly(2-methacryloyloxyethyl dihydrogen phosphate-co- -isopropylacrylamide), poly(N-ethyl-4-vinyl-pyridinium bromide), and poly(N-isopropyl-acrylamide-co-acrylic acid).

33. A method of delivering an ionic macromolecule to an individual in need thereof comprising administering to the individual a microsphere encapsulating an ionic complex of the ionic macromolecule and a polyelectrolyte-poly(ethylene glycol)diblock copolymer.

34. A method of increasing transfection efficiency of DNA into mammalian cells, the method comprising:
(a) forming an ionic complex comprising the DNA and a cationic polyelectrolyte-poly(ethylene glycol)diblock copolymer; and
(b) incubating the ionic complex with the mammalian cells in the presence of a transfection enhancer such that the ionic complex, and hence the DNA, enters the mammalian cells.

35. The method of claim 34 wherein the cationic polyelectrolyte-poly(ethylene glycol)diblock copolymer comprises polyhistidine-poly(ethylene glycol)diblock copolymer.

36. The method of claim 34 wherein the transfection enhancer comprises a cationic lipid.

37. A method for increasing efficiency of incorporation of ionic macromolecules into microspheres, the method comprising:
(a) determining the charge of the ionic macromolecules;
(b) forming ionic complexes comprising the ionic macromolecules and a polyelectrolyte-poly(ethylene glycol)diblock copolymer of opposite charge; and
(c) mixing the ionic complexes with a polymer suitable for forming microspheres to form a mixture, and treating the mixture for formation of microspheres.

38. The method of claim 37 wherein the polymer suitable for forming microspheres comprises poly(lactide-co-glycolide).

39. The method of claim 37 wherein treating the mixture for formation of microspheres comprises forming a water-in-oil-in-water emulsion and solvent evaporation.
